(19) United States
(12) Patent Application Publication
(10) Pub. No.: US 2018/0095061 A1
(43) Pub. Date: Apr. 5, 2018

(54) CO DETECTOR ADAPTER AND MOBILE DEVICE APPLICATION

(71) Applicant: Universal Enterprises, Inc., Beaverton, OR (US)

(72) Inventors: Michael John Kane, Portland, OR (US); Sean Patrick Tierney, Milwaukie, OR (US)

(73) Assignee: Universal Enterprises, Inc., Beaverton, OR (US)

(21) Appl. No.: 15/283,302

(22) Filed: Oct. 1, 2016

(51) Int. Cl. G01N 33/00

(52) U.S. Cl. CPC G01N 33/004; H04W 88/02; G01N 33/0062

(57) ABSTRACT

A gas detector adapter for a mobile device having a housing, a inlet on the housing through which ambient air is able to pass for detection of a gas such as carbon monoxide by a carbon monoxide detection sensor, an electrical plug extending rearward from the housing configured to electrically interconnect with an audio jack on the mobile device, and electronic circuitry adapted for transmitting sensor detection signals from the sensor through the mobile device audio jack so that application software downloaded and running on the mobile device is able to convert the transmitted sensor detection signals into digital data for display on the mobile device display.

### CROSS-REFERENCE TO RELATED APPLICATIONS

None.

### BACKGROUND OF THE INVENTION

The technical field of invention relates to an adapter and mobile device application for detecting the presence of a gas. More particularly, the present invention pertains to carbon monoxide (CO) detector, or detector of a particular target gas, adapter and mobile device application for detecting the presence and intensity of CO or a target gas, the device and associated application software ideally suited for use in the heating, ventilating, and air conditioning and refrigeration (HVAC, or HVAC/R) industry.

Existing gas detection instruments used in the HVAC industry include the Eagle and Smart Bell Plus combustion meters manufactured and distributed by UEi. The Fyrite INSIGHT combustion gas analyzer by Bacharach is another existing handheld-sized instrument. Yet another existing combustion analyzer device is the Testo 330 Flue Gas analyzer. And still other existing combustion gas analyzers include the BTU900 and BTU4400 by E Instruments.

In all of the existing devices, the device comprises a standalone instrument that is not integrally operative with a mobile device such as a smartphone. Such standalone instrument devices also comprise CO gas sensors that require periodic calibration or sensor replacement. Although the life of the sensors used in such devices is improving over time (with improvements in the sensors being used) and in-field replacement procedures are becoming more readily available, sensor calibration and sensor performance varies widely from device to device and depend upon the gas type being sensed and the technologies of the sensors used. Different manufacturers use different sensor arrangements and technologies. Some use conventional electrochemical Oxygen and CO sensors, and others use an electro-optical CO2 sensor to eliminate the O2 sensor altogether (and, thus, eliminate the costs associated with its replacement or recalibration). Still other designs use different sensor technologies, for example catalytic (or Pellistor), non-dispersive infrared (NDIR), thermal conductivity, solid state/semiconductor, or standard/conventional electrochemical type sensors. Each different technology and each different type of gas to be sampled and measured typically requires its own unique physical structure and electronic (metering) circuitry, further complicating the tasks of HVAC field technicians.

The CO71A carbon monoxide detector made by UEi is used for ambient testing (i.e. not for in-flue or in warm air streams testing) to monitor CO levels in commercial and residential living spaces, warehouse operations, combustion engine repair facilities, public facilities, and any other indoor areas where people may work or live. The C071A comprises a handheld-sized device with preset alarms with three-color warning light (green when CO is 2-9 ppm, amber when CO is 10-35 ppm, and red when CO is higher), audible alert, maximum CO detection capture (for displaying the highest concentration (in ppm's) during continuous detection), a CO detection range from zero to 999 ppm, and an approximate CO (electrochemical) sensor life of five years with calibration of the CO detection sensor recommended annually.

Each of the existing gas detection device designs has disadvantages in terms of cost, complexity of design, ease of use, physical dimensions of the device, method of measurement data collection, method for providing alerts or alarms, form factor and ergonomics of the device, design aesthetics, and/or other factors. What is needed are designs for a gas detection attachment and associated application software for a mobile device that address one or more disadvantage of existing gas detection device designs.

The foregoing and other objectives, features, and advantages of the invention will be more readily understood upon consideration of the following detailed description of the invention taken in conjunction with the accompanying drawings.

### BRIEF DESCRIPTION OF THE SEVERAL DRAWINGS

For a more complete understanding of the present invention, the drawings herein illustrate examples of the invention. The drawings, however, do not limit the scope of the invention. Similar references in the drawings indicate similar elements.

FIG. 1 illustrates a user's hand holding a mobile device with a gas detector attachment and running application software for operating the attachment, according to preferred embodiments.

FIG. 2 illustrates a gas detector audio jack adapter, according to preferred embodiments.

FIG. 3 illustrates a block diagram for operation of a gas detector attachment and mobile device with application software, according to preferred embodiments.

FIGS. 4A and 4B comprise schematics of exemplary circuitry comprising the attachment of FIG. 2, according to various embodiments.

FIG. 5 is an illustration of connecting a clamp head adapter to a clamp meter, according to various embodiments.

FIG. 6 illustrates a gas detector adapter connected to a clamp head adapter and clamp meter, according to various embodiments.

FIG. 7 illustrates different visual display features of a gas detection adapter connected to a mobile device running an associated software application for use with the gas detection adapter and smartphone combination, according to preferred embodiments.

### DETAILED DESCRIPTION OF PREFERRED EMBODIMENTS

In the following detailed description, numerous specific details are set forth in order to provide a thorough understanding of the preferred embodiments. However, those skilled in the art will understand that the present invention may be practiced without these specific details, that the present invention is not limited to the depicted embodiments, and that the present invention may be practiced in a variety of alternate embodiments. In other instances, well known methods, procedures, components, and systems have not been described in detail.

Preferred embodiments comprise: a mobile communications device, or smartphone, attachment having gas detector sensor means, circuitry for receiving power from the mobile device, circuitry for providing gas detector sensor signals to the mobile device, and the attachment connected to the mobile device via the mobile device audio jack (socket), or, alternatively, the mobile device charging/data port such as the mini-USB port for Android or similar devices or lightning charging/data port for IOS devices; and mobile device application software downloaded onto the mobile device and adapted to allow a user of the mobile device with gas detector sensor attachment to display, store, retrieve, graph, and manipulate gas detector measurement data. Although the preferred embodiments are depicted herein in the context of a CO detector adapter comprising sensor means for detecting carbon monoxide (CO) gas, sensor means for detecting a different gas may be used and the preferred embodiments may be characterized more generally as comprising a gas detector adapter with means for detecting a particular gas.

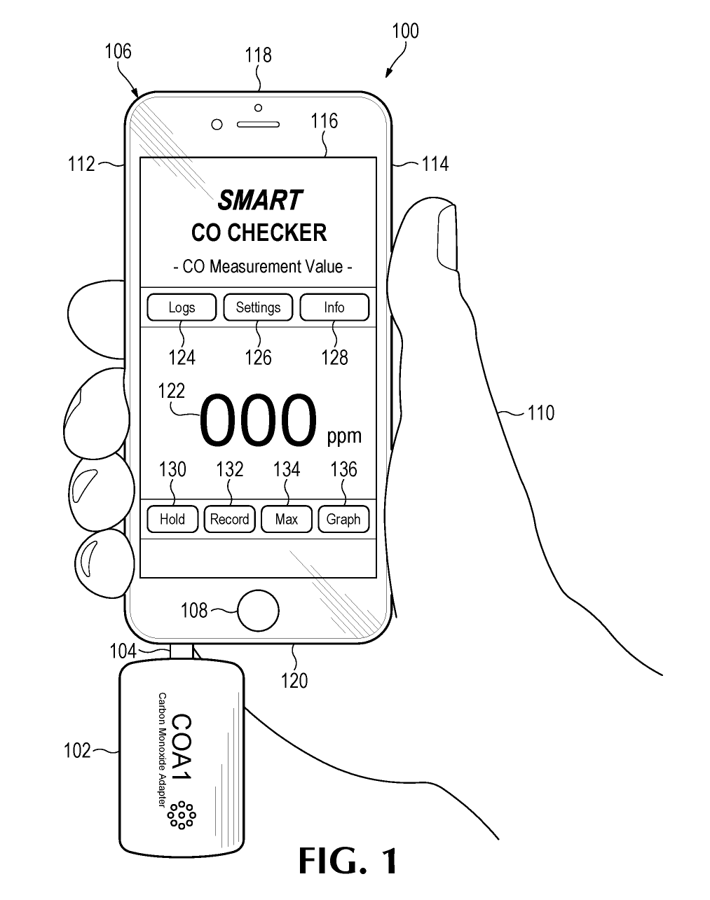

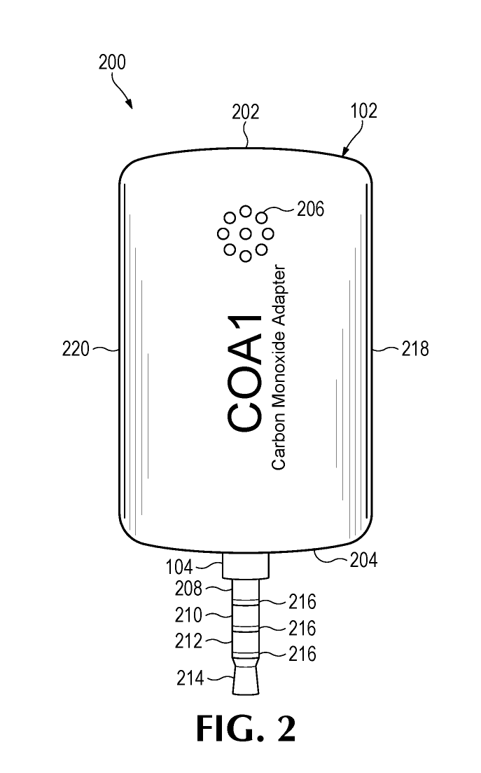

A preferred embodiment is shown in FIGS. 1 and 2, with FIG. 1 showing an CO detector attachment (or adapter) 102 connected to the mobile device 106 audio jack, and FIG. 2 showing the CO detector attachment 102 having an inlet grille 206 and an inwardly extending electrical interface plug 104 for connecting into the audio jack of the mobile device 106. As will be discussed further, alternative embodiments may implement the CO detector adapter 102 using different shapes/dimensions for the housing or, for example, orienting the inlet grille 206 differently (such as directing the inlet grille 206 on a different face of the adapter housing).

FIG. 1 illustrates a preferred orientation 100 with a user's hand 110 holding the mobile device 106 with a CO detector attachment 102 connected via the attachment plug 104 and the device audio jack, and running application software downloaded to the mobile device for operating the attachment. The attachment 102 is shown with a plug 104 inserted into the audio jack of the mobile device 106. The mobile device 106 shown is an iPhone 5 style smartphone with its main front face button 108 oriented so that the audio jack and plug 104 axis is directed inward toward the user's hand 110 for testing ambient air for detection of CO gas. If the mobile device 106 were illustrated as an iPhone 4 style smartphone, the audio jack and plug 104 axis would be directed outward extending from the far end 118 of the device 106 in a direction away from the user's hand 110, and the CO detector adapter 102, when connected to the mobile device 106, would extend outward from the far end 118 of the device 106.

The mobile device 106 is shown having a length dimension between a near end 120 and a far end 118, and a width dimension between a left side 112 and a right side 114. A thickness dimension, not shown, is the distance between the front of the phone/front of display 116 and the backside of the phone. The thickness dimension is perpendicular to the plane formed by the length and width dimensions. The main camera optics of an iPhone 5 are on the backside of the phone and are directed away from the front face of the display 116 and in a direction parallel with the thickness dimension of the phone (and perpendicular to the plane defined by the length and width dimensions).

Preferably, the user holds the mobile device 106 as shown in FIG. 1, with the CO detector adapter 102 connected into the audio jack of the device 106, with the inlet grille 206 of the CO detector adapter 102 oriented so as to be exposed to the ambient air to be tested and monitored. The user preferably opens an app downloaded onto the mobile device for operating the CO detector attachment 102. Using the touch screen/display 116, the user is preferably presented with simple options for operating the attachment 102. For example, and as shown, opening the (“CO Checker”) app presents the user with several buttons, including “Logs” 124, “Settings” 126, “Info” 128, “Hold” 130, “Record” 132, “Max” 134, and “Graph” 136. Once the app opens and establishes communications with the attachment 102, real time measurement information 122 is preferably displayed. If the real time measurement information 122 is, for example, zero parts-per-million (ppm), then the measurement information 122 reads “000 ppm”. As the user moves the attachment 102 to another place for ambient gas detections, or as concentration of gas being exposed to the inlet 206 changes, the measurement information 122 changes to display the updated sensed and calculated gas concentration measurement.

The app software buttons shown on display 116 in FIG. 1 preferably comprise buttons on the app main screen, and the buttons are preferably customizable by the user when downloading and initially setting up the app and during subsequent use of the app. Each button preferably provides the user with quick access to a particular app function. Selecting “Logs” 124 preferably causes retrieval and display of previously recorded CO concentration measurements. “Settings” 126 preferably provides the user with display options such as font size, background display options, information to include with recorded measurements (such as date formats, location information, client/job information, etc.), and other options; and the available settings preferably includes options for the user to customize the buttons displayed on display 116. For example, a “Min” button might be available if the user would like to keep track of minimum measurement values and have the “Min” button added (i.e. pinned) to the app main display screen. Further, the “Settings” 126 button preferably provides, when selected, listed options that the user may scroll through using standard finger swipe motions on touch screen/display 116. “Info” 128 may provide information about the amount of data saved, remaining memory available, software version information, etc. “Hold” 130 preferably retains the presently displayed measurement information on the display 116. “Record” 132 preferably saves the measurement or series of measurements into memory. “Max” 134 preferably presents the highest measurement value for a particular series of measurements. And “Graph” preferably presents a series of measurements graphically on display 116.

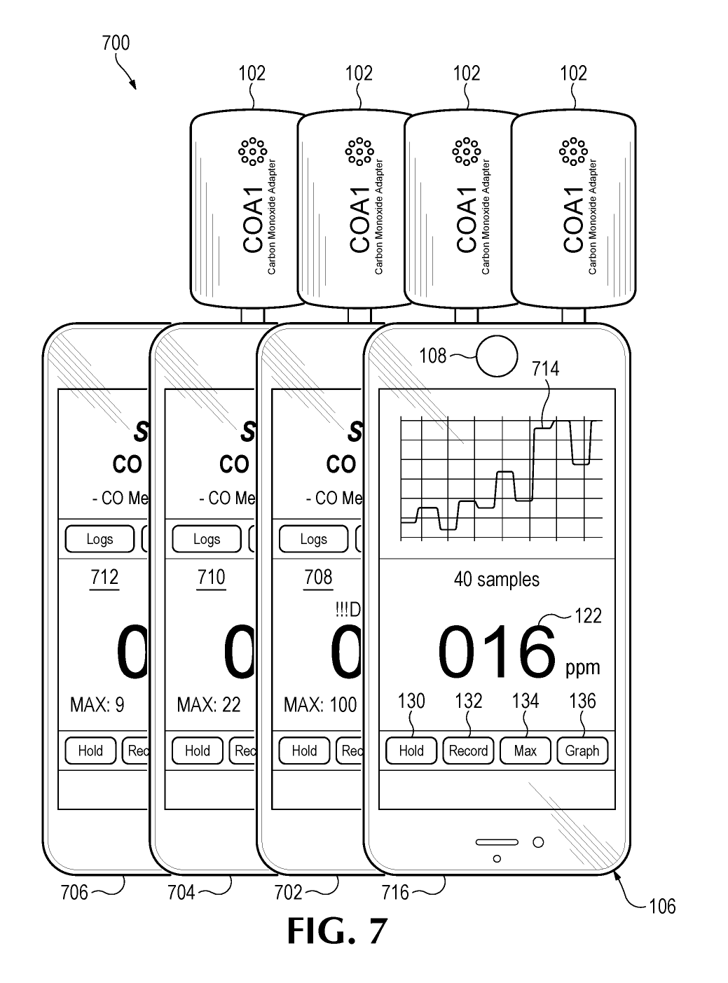

An exemplary graph 714 is shown in FIG. 7, which also illustrates different visual display features 700 of a gas detection adapter 102 connected to a mobile device 106 running an associated software application for use with the gas detection adapter and smartphone combination, according to preferred embodiments. The graph 714 on the phone 716, in this example, shows a graph 714 of 40 samples and the most recent real time measurement 122 of “016 ppm” as the CO concentration detected by the connected CO detector adapter 102. Phone 702 depicts an example display 708 alerting with a red colored screen, indicating a max CO detected concentration of “100 ppm” and, for additional emphasis, an indication of “!!!DANGER!!!”. Phone 704 depicts an example display 710 alerting with a yellow colored screen and indicating a max CO concentration of “22” ppm. Phone 706 depicts an example display 712 alerting with a green colored screen and indicating a max CO concentration of “9ppm”.

Referring back to FIG. 2, a CO detector audio jack adapter 200 is illustrated, according to preferred embodiments. The CO detector adapter 200 preferably comprises attachment 102 having a (housing) length dimension between a far end 202 extending away from the attachment (adapter) plug 104 and a near/rear end 204, and a width dimension between a left side 220 and a right side 218. A thickness dimension is the distance between a front side of the housing depicted as having the inlet grille 2016 positioned thereon and a backside of the housing opposite the front side. The thickness dimension is perpendicular to the plane formed by the length and width dimensions. Preferably, the length of the attachment 102 is greater than its width, and the length of the attachment 102 is greater than its thickness. Preferably, the dimensions of the attachment 102 are as small as possible. The length of the attachment 102 is preferably less than the length of the mobile device 106, and is preferably less than the width of the mobile device 106. The width of the attachment 102 is preferably less than the width of the mobile device 106, and is preferably (considerably) less than the length of the mobile device 106. Preferably, just as the length of the mobile device is (preferably) greater than either of its width or thickness dimensions, the length of the attachment 102 is greater than either of its width or thickness dimensions.

In one embodiment, the housing portion of attachment 102 comprises a rectangular prism with a cross-sectional area (defined by its width and thickness) along its full length from far end 202 to its near end 204. In one embodiment, the housing width and thickness dimensions are approximately equal. In a preferred embodiment, the housing is a rectangular prism with rounded sides such that the front and back sides have flattened areas and the sides are more rounded. The resulting rounded rectangular prism preferably has a width dimension slightly greater than its thickness, due to the flattened front and back areas. In other alternative embodiments, the attachment 102 housing comprises a nearly cylindrical shape. In the embodiment shown in FIGS. 1 and 2, attachment 102 housing comprises an oval prism shape, with a cross-section having rounded left and right sides separated by a flat front and back sides, the cross-section extending outward from a near end 204 to a far end 202.

Extending rearward from the attachment 102 housing is, as shown in FIG. 2, an electrical interface plug 104 having dimensional characteristics to electrically and structurally cooperatively insertingly mate into the audio jack of a mobile device 106. The plug 104 preferably comprises electrical conductors 208, 210, 212, and 214, each separated by an electrical insulator 216. A standard audio jack typically includes electrical conductor configured for receiving the corresponding conductors on plug 104, with plug conductor 208 corresponding to an audio jack conductor for the mobile device microphone or MIC; plug conductor 210 corresponding to an audio jack conductor for ground; plug conductor 212 corresponding to an audio jack conductor for right audio channel signal; and plug conductor 214 corresponding to an audio jack conductor for left audio channel signal. The electrical conductors 208, 210, 212, and 214 are available for use by the mobile device application software and circuitry comprising the attachment 102 for providing power to the attachment 102 circuitry therein, and for transferring data and sensor signals for operation of the attachment 102.

In other preferred embodiments, not shown, the plug 104 may instead comprise a male connector for use with an IOS lightning charger/data port or an Android mini-USB, or any other electrical interface with a mobile device 106. The available conductors on IOS lightning, mini-USB, or similar connectors may be used in similar fashion as the electrical conductors 208, 210, 212, and 214 shown in FIG. 2 with sensor circuitry and supporting signal processing circuitry comprising attachment 102.

In preferred embodiments, the attachment 102 includes a battery, such as a 3V battery. In other embodiments, the attachment 102 has no battery and is powered by the mobile device via electrical conductors such as electrical conductors 208, 210, 212, and 214.

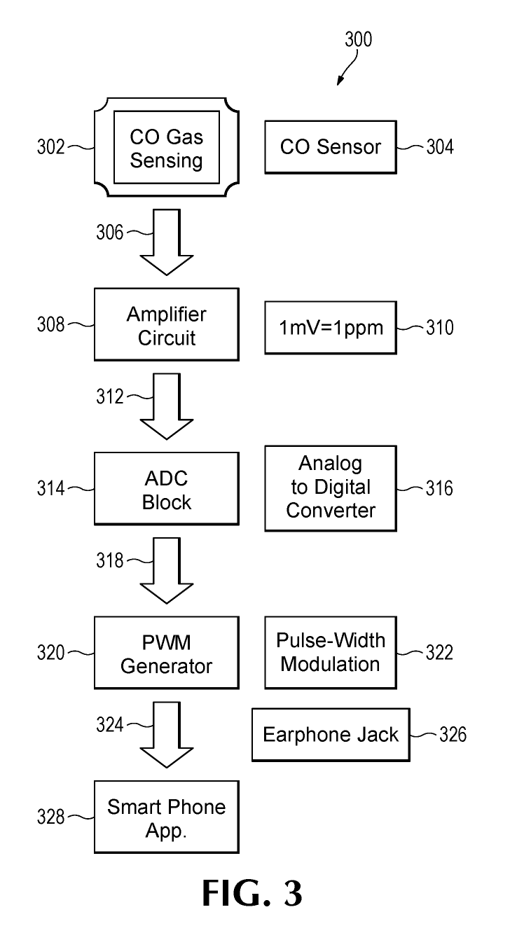

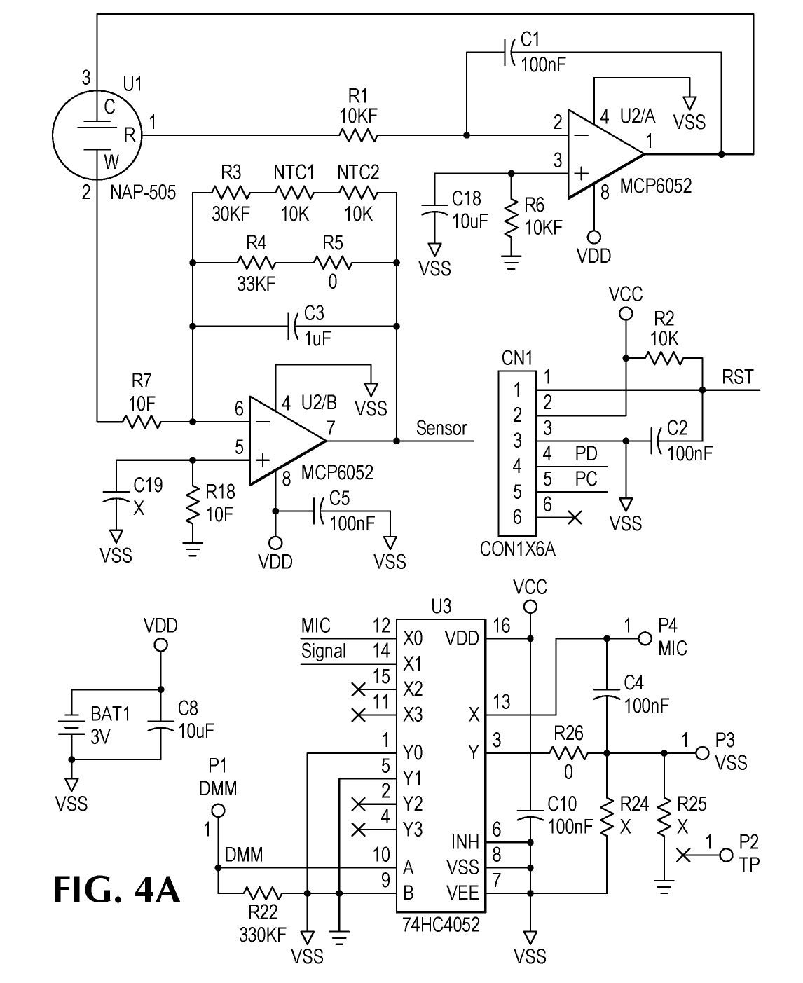

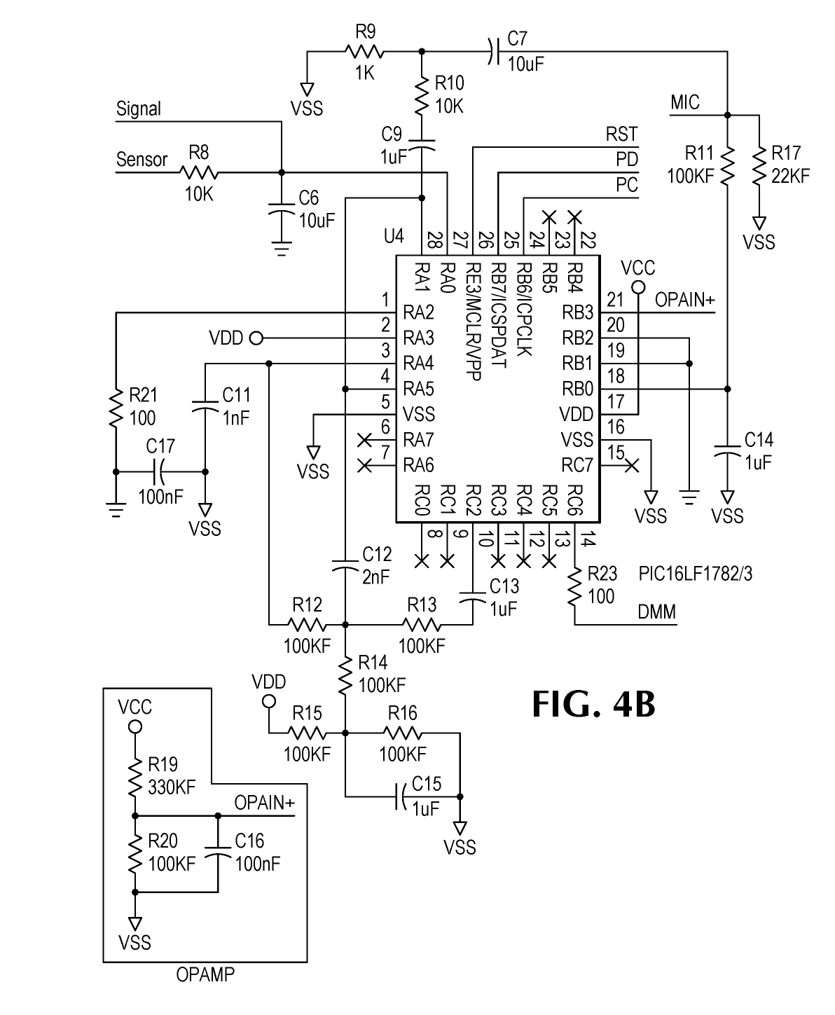

FIGS. 3, 4A and 4B provide exemplary implementation of the CO detector adapter 200 and mobile device application for the preferred orientation 100 as illustrated in FIGS. 1 and 2. FIG. 3 illustrates a block diagram 300 for operation of a CO detector attachment and mobile device with application software, according to preferred embodiments, and FIGS. 4A and 4B comprise schematics of exemplary circuitry comprising the attachment of FIG. 2, according to various embodiments. In one embodiment, in a CO gas sensing step 302, a CO sensor 304 detects the concentration of CO gas in ambient air exposed through the inlet grille 206. The detected signals are then sent 306 to an amplifier circuit 308 for amplification. Preferably, each 1 ppm is 1 millivolt (mV) in detected signal strength. In one embodiment, a maximum of 1000 ppm corresponds to 1000 mV detected signal strength. The amplified detected signal is then sent 312 to an analog-to-digital converter (ADC) block 314 of a microcontroller (MCU). The analog signals measured by the ADC block 314 are converted to a digital signal 316, and then the digital signal/data is sent 318 to a pulse-width modulation (PWM) generator 320. The PWM generator 320 converts the digital data to audio signals which are received 324 through the audio (or earphone) jack 326. Finally, the audio signals received through the earphone jack 326 are converted to digital data by the app program 328 for display (of the gas detection measurement data) on the smartphone/mobile device.

The application software preferably comprises a mobile device app for use with the CO detection adapter that includes programming instructions downloadable for storage and execution on the mobile device 106 and adapted to transform sensor detection signals received through the audio jack of said mobile device from pulse-width modulation signals generated by circuitry of the CO detector adapter 102 to digital data for display of gas detection measurement information on the display screen 116 of the mobile device 106. The programming instructions preferably enable use of the mobile device touch screen for receiving user selection (eg. by touching a virtual button displayed on the touch screen) of user-selectable and user-customizable options for such things as visual display preferences (eg. font sizes), whether to initiate or stop recording temperature measurements, and to toggle on and off display of graphed gas detection measurements.

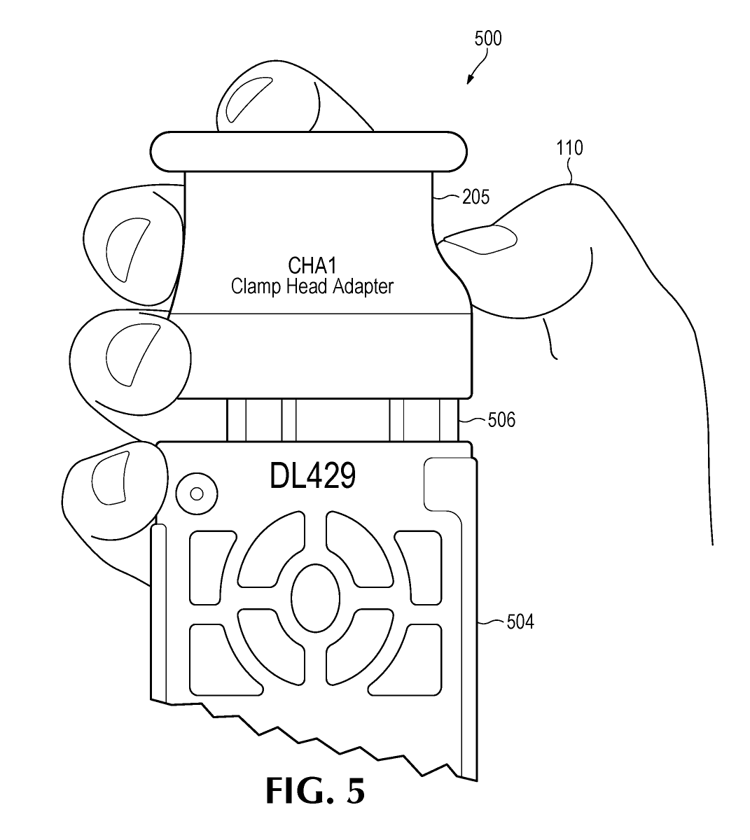

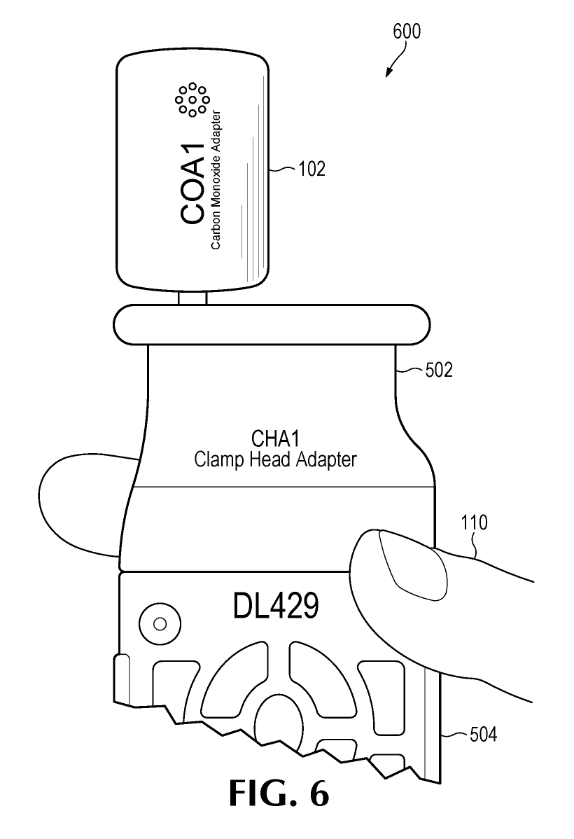

In preferred embodiments, the CO detector adapter 102 may be used with test and measurement equipment having an audio jack socket. FIG. 5 is an illustration 500 of connecting a clamp head adapter 502 to a clamp meter 504, according to various embodiments. The clamp head adapter 502 preferably comprises electrical connectors oriented and configured to receive the connections 506 of a clamp meter body 504 similar to the DL429 clamp meter body manufactured by UEi shown. The clamp head adapter 502, as illustrated, may be attached to the clamp meter body 504 using a hand 110 (or two, not shown). As shown in FIG. 6, once the clamp head adapter 502 is connected to the clamp meter body 504, a CO detector adapter 102 may be connected into a electrical plug in the clamp head adapter 502 so that the CO detector adapter 102 functions substantially as described above but with the clamp meter 504 providing power and receiving detected, amplified, and converted signals instead of a mobile device 106. The clamp head adapter 502 preferably allows a CO detector adapter 102 to be used with compatible clamp meters. In alternative embodiments, the clamp head adapter 502 may comprise an adapter that accepts a CO detector adapter 102, as shown in FIG. 2, and connects to a more generally applicable test and measurement instrument body that accepts multiple different measurement heads.

In one embodiment, the CO detector adapter 102 allows for monitoring CO concentration on Android or IOS smartphones, or on UEi clamp meters (such as models DL429, DL389, DL379B, and DL379) when coupled with a clamp head adapter (such as model CHA1). The CO detector adapter 102 preferably detects CO concentration in the range of zero to 999 ppm, with a resolution of 1 ppm, with an accuracy +/−3% of reading +1 ppm, and in ambient temperatures of 32 degrees F. to 104 degrees F. The application software downloadable to a smartphone and having an audio jack suitable for use with the CO detector adapter 102, preferably allows logging and emailing capabilities, so that saved data may be collected and exported via email. In preferred embodiments, the CO detector adapter 102 comprises a CO sensor that self-calibrates when the CO detector adapter 102 is connected to a smartphone through the smartphone's earphone jack, with application software operating on the connected smartphone that presents CO detection readings in a numeric and graphical manner in accordance to preset industry safety thresholds or responsive to user customizable alarm level settings.

The terms and expressions which have been employed in the foregoing specification are used therein as terms of description and not of limitation, and there is no intention in the use of such terms and expressions of excluding equivalents of the features shown and described or portions thereof, it being recognized that the scope of the invention is defined and limited only by the claims which follow.

What is claimed is:

1. A gas detector adapter for a mobile device comprising:
a housing having a length between a far end of said housing and a near end of said housing;
an inlet on a face of the housing, through which ambient air is able to pass to a gas detection sensor within said housing;
an electrical plug extending rearward from the near end of the housing, the electrical plug configured to electrically interconnect with an audio jack on said mobile device; and
circuitry adapted for transmitting sensor detection signals from said gas detection sensor through said audio jack on said mobile device so that application software running on said mobile device is able to convert the transmitted sensor detection signals into digital data for display on said mobile device.

2. The adapter of claim 1 wherein said gas detection sensor is a carbon monoxide gas detection sensor.

3. The adapter of claim 2 wherein said circuitry comprises amplifier circuitry to amplify output from said gas detection sensor.

4. The adapter of claim 3 wherein said circuitry comprises an analog-to-digital converter to convert amplified output from said gas detection sensor to digital data.

5. The adapter of claim 4 wherein said circuitry comprises a pulse-width modulation generator to convert said digital data to audio jack signals for sending sensor detection signals to said mobile device via said electrical plug.

6. A mobile device application for use with a gas detector adapter comprising programming instructions downloadable for storage and execution on said mobile device and adapted to transform sensor detection signals received through an audio jack of said mobile device from pulse-width modulation signals generated by circuitry of said gas detector adapter to digital data for display of gas concentration measurement information on a display screen of said mobile device.

7. The application of claim 6, wherein said programming instructions enable touch screen means for user selection of options for visual display, recording, and graphing said gas concentration measurement information.

8. The application of claim 7, wherein said user selection is made by touching a button presented on said touch screen.

9. The application of claim 1, wherein said mobile device comprises an IOS or Android type smartphone.

10. The adapter of claim 1 wherein said gas detector adapter includes a carbon monoxide gas detection sensor for detecting a concentration of carbon monoxide gas.

11. A method of measuring a gas concentration in ambient air comprising:
providing a gas detector adapter for a mobile device including a housing having a length between a far end of said housing and a near end of said housing, an inlet on a face of the housing through which ambient air is able to pass to a gas detection sensor within said housing, an electrical plug extending rearward from the near end of the housing and configured to electrically interconnect with an audio jack on said mobile device, and circuitry adapted for transmitting sensor detection signals from said gas detection sensor through said audio jack on said mobile device so that application software running on said mobile device is able to convert the transmitted sensor detection signals into digital data for display on said mobile device;
providing a mobile device having an audio jack;
downloading said application software to said mobile device;
plugging the electrical plug of the gas detector adapter into the audio jack of the mobile device; and
running said application software on said mobile device.

12. The method of claim 11 further comprising:
sensing a concentration of a gas using a gas detection sensor in said gas detector adapter;
amplifying detection signals from said gas detection sensor;
converting the amplified detection signals to digital signals;
converting the digital signals to pulse-width modulated audio signals;
transmitting the pulse-width modulated audio signals to mobile device via the electrical plug of the gas detector adapter and the mobile device audio jack; and
displaying the gas concentration measurement using said downloaded and running application software.

12. The method of claim 11 wherein said mobile device comprises an IOS or Android type smartphone.

13. The method of claim 11 wherein said gas detection sensor is a carbon monoxide gas detection sensor.
